(19) United States
(12) Patent Application Publication
(10) Pub. No.: US 2024/0398470 A1
(43) Pub. Date: Dec. 5, 2024

(54) ALL IN ONE BASILICA SYSTEM

(71) Applicant: BOSTON SCIENTIFIC SCIMED, INC., MAPLE GROVE, MN (US)

(72) Inventors: Daire Grant, Galway (IE); Tim O'Connor, Galway (IE); Joseph Murphy, Galway (IE); Declan Loughnane, Galway (IE)

(73) Assignee: BOSTON SCIENTIFIC SCIMED, INC., MAPLE GROVE, MN (US)

(21) Appl. No.: 18/678,249

(22) Filed: May 30, 2024

Related U.S. Application Data
(60) Provisional application No. 63/470,678, filed on Jun. 2, 2023.

(51) Int. Cl. A61B 18/14; A61B 18/00

(52) U.S. Cl. CPC A61B 18/1492; A61B 2018/00166; A61B 2018/00184; A61B 2018/00369; A61B 2018/00601; A61B 2018/144

(57) ABSTRACT

A basilica system for use in replacement heart valve implant procedures may include a first catheter including a side port disposed proximal of a distal tip, a conductive guidewire slidably disposed within the first catheter and configured to be advanced out the side port proximate a leaflet of a replacement heart valve implant disposed within a native heart valve of a patient, a second catheter including a lumen extending to a distal end of the second catheter, and a snare shaft slidably disposed within the second catheter, the snare shaft including a wire loop configured to grasp the conductive guidewire. The basilica system may include a delivery catheter. The first and second catheters may be disposed within the delivery catheter. The conductive guidewire may be configured to be electrically coupled to a source of RF energy. The conductive guidewire may be configured to puncture the leaflet.

### CROSS-REFERENCE TO RELATED APPLICATIONS

This application claims the benefit of priority of U.S. Provisional Application No. 63/470,678 filed Jun. 2, 2023, the entire disclosure of which is hereby incorporated by reference.

### TECHNICAL FIELD

The disclosure relates generally to medical devices and more particularly to medical devices pertaining to replacement heart valve implants and procedures related thereto.

### BACKGROUND

A wide variety of intracorporeal medical devices have been developed for medical use including, artificial heart valves for repair or replacement of diseased heart valves. Diseases and/or medical conditions that impact the cardiovascular system are prevalent throughout the world. Some relatively common medical conditions may include or be the result of inefficiency, ineffectiveness, or complete failure of one or more of the valves within the heart. Treatment of defective heart valves poses other challenges in that the treatment often requires the repair or outright replacement of the defective valve. As improvements are made to devices and procedures, replacement heart valve implants are being implanted into younger and lower risk patients who in turn have a longer life expectancy. In some cases, these patients may outlive the useful lifespan of their initial replacement heart valve implant.

In such situations, a second replacement heart valve implant procedure may be required or desired. The second replacement heart valve implant may be implanted directly within the original/initial replacement heart valve implant. However, such an arrangement may present additional risks related to access to the coronary arteries. As patients age, the need to place stents in the coronary arteries is not uncommon. It is possible that the coronary ostia may become partially or completely obstructed by the leaflets of the original/initial replacement heart valve implant when the second replacement heart valve implant is implanted therein, thereby limiting access and/or making access more difficult for treatment of and/or within the coronary arteries. In some cases, this can be a life-threatening complication, particularly in patients with low-lying coronary ostia and/or inadequate aortic sinus width. In some cases, due to the shape and/or anatomical configuration of the aortic sinus (e.g., aortic sinus width, etc.), blood may still flow to the coronary arteries even if/when the original/initial replacement heart valve implant is held open against the coronary sinus by the second replacement heart valve implant. The risk may be exacerbated in valve-in-valve procedures because the leaflets of the original/initial replacement heart valve implant may sit higher in the aortic sinus that the native valve leaflets, and thus would be more likely to partially or completely obstruct the coronary ostia.

When treating the coronary arteries, the coronary ostia are accessed from within the replacement heart valve implant(s). If the leaflets of the original/initial replacement heart valve implant partially or completely obstruct access to the coronary arteries from inside the replacement heart valve implant(s), locating and/or entering the coronary ostia may be more difficult and/or may require additional time, thereby increasing risk to the patient.

A leaflet splitting procedure known as “basilica” has been developed that modifies the leaflets of the original/initial replacement heart valve implant prior to implantation of the second replacement heart valve implant to improve access to the coronary ostia. However, the basilica procedure currently has no easy-to-use dedicated device designed therefor and may be very challenging to perform accurately using “standard” devices. There is an ongoing need to provide alternative medical devices and/or systems as well as alternative methods for manufacturing and using medical devices and/or systems.

### SUMMARY

In one example, a basilica system for use in replacement heart valve implant procedures may comprise a pigtail catheter comprising a side port disposed proximal of a distal tip of the pigtail catheter, a conductive guidewire slidably disposed within a first lumen of the pigtail catheter and configured to be advanced out the side port proximate a leaflet of a replacement heart valve implant disposed within a native heart valve of a patient, a snare catheter comprising a lumen extending to a distal end of the snare catheter, and a snare shaft slidably disposed within the lumen of the snare catheter, the snare shaft comprising a wire loop extending from a distal end of the snare shaft and configured to grasp a distal end of the conductive guidewire.

In addition or alternatively to any examples disclosed herein, the pigtail catheter may further comprise a second lumen extending proximally from the distal tip of the pigtail catheter.

In addition or alternatively to any examples disclosed herein, the first lumen extends proximally from the side port.

In addition or alternatively to any examples disclosed herein, the pigtail catheter includes a preformed recurve portion proximal of the distal tip of the pigtail catheter in an unconstrained configuration.

In addition or alternatively to any examples disclosed herein, the side port is disposed proximate a base of the preformed recurve portion.

In addition or alternatively to any examples disclosed herein, a centerline of the preformed recurve portion is generally disposed within a plane and the side port opens generally perpendicular to the plane.

In addition or alternatively to any examples disclosed herein, a distal portion of the snare catheter includes a pre-shaped bend in an unconstrained configuration.

In addition or alternatively to any examples disclosed herein, the pre-shaped bend may be configured to orient the distal end of the snare catheter toward the side port of the pigtail catheter.

In addition or alternatively to any examples disclosed herein, the pre-shaped bend orients the distal end of the snare catheter at an oblique angle relative to a longitudinal axis of the snare catheter.

In addition or alternatively to any examples disclosed herein, the basilica system may further comprise a delivery catheter.

In addition or alternatively to any examples disclosed herein, the pigtail catheter and the snare catheter are slidably disposed within the delivery catheter.

In addition or alternatively to any examples disclosed herein, the basilica system may further comprise a source of RF energy electrically coupled to the conductive guidewire.

In addition or alternatively to any examples disclosed herein, a basilica system for use in replacement heart valve implant procedures may comprise a delivery catheter, a first catheter slidably disposed within the delivery catheter, a second catheter slidably disposed within the delivery catheter, a conductive guidewire slidably disposed within the first catheter and configured to be electrically coupled to a source of RF energy, the conductive guidewire including a distal end configured to puncture a leaflet of a replacement heart valve implant, and a snare shaft slidably disposed within a lumen of the second catheter, the snare shaft comprising a wire loop extending from a distal end of the snare shaft and configured to grasp the distal end of the conductive guidewire.

In addition or alternatively to any examples disclosed herein, in a deployed configuration, the first catheter comprises a preformed recurve portion disposed proximal the distal tip, and an elongate portion extending proximally from the preformed recurve portion.

In addition or alternatively to any examples disclosed herein, the first catheter comprises a side port disposed in the preformed recurve portion of the first catheter when the first catheter is in the deployed configuration.

In addition or alternatively to any examples disclosed herein, the preformed recurve portion is shaped and configured to align with a cusp of the leaflet of the replacement heart valve implant.

In addition or alternatively to any examples disclosed herein, in a deployed configuration, the second catheter includes a distal portion having a pre-shaped bend configured to orient a distal end of the second catheter toward the side port.

In addition or alternatively to any examples disclosed herein, when the first catheter is in the deployed configuration and the second catheter is in the deployed configuration, distal advancement of the delivery catheter relative to the first catheter and the second catheter urges the distal end of the second catheter closer to the side port formed in the first catheter than to the distal tip of the first catheter.

In addition or alternatively to any examples disclosed herein, when the distal end of the second catheter is in close proximity to the side port, the side port is configured to direct the conductive wire toward the wire loop when the conductive wire is advanced distally relative to the first catheter.

In addition or alternatively to any examples disclosed herein, a method of splitting a leaflet of a replacement heart valve implant disposed within a native heart valve of a patient may comprise: advancing a delivery catheter to a position adjacent the replacement heart valve implant; advancing a first catheter distally out of the delivery catheter to a position adjacent an outer surface of the leaflet; advancing a second catheter distally out of the delivery catheter to a position adjacent an inner surface of the leaflet; advancing the delivery catheter distally relative to the first catheter and the second catheter to urge a distal end of the second catheter toward a side port formed in the first catheter proximal of a distal tip of the first catheter; advancing a conductive guidewire out of the first catheter and through the leaflet at a puncture site; grasping the conductive guidewire with a wire loop extending from the second catheter; and pulling the conductive guidewire through the leaflet to lacerate the leaflet from the puncture site to a free edge of the leaflet.

In addition or alternatively to any examples disclosed herein, the method may further comprise applying RF energy to the conductive guidewire while pulling the conductive guidewire through the leaflet to lacerate the leaflet.

In addition or alternatively to any examples disclosed herein, advancing the conductive guidewire out of the first catheter and through the leaflet includes advancing a distal end of the conductive guidewire into the wire loop.

In addition or alternatively to any examples disclosed herein, the first catheter comprises a preformed recurve portion disposed proximal the distal tip in a deployed configuration, and wherein advancing the first catheter distally out of the delivery catheter to the position adjacent the outer surface of the leaflet further comprises positioning the preformed recurve portion in a cusp of the leaflet with the side port facing towards the leaflet.

In addition or alternatively to any examples disclosed herein, the method may further comprise, prior to pulling the conductive guidewire through the leaflet to lacerate the leaflet, retracting the wire loop into the second catheter to pull a distal end of the conductive guidewire into the second catheter.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a partial cutaway view illustrating selected aspects of a replacement heart valve implant positioned within a native valve annulus of a heart;

FIG. 2 is a partial cutaway view illustrating selected aspects of a replacement heart valve implant positioned within a native valve annulus of a heart;

FIG. 3 is a schematic view illustrating selected aspects related to implanting a second replacement heart valve implant within a first replacement heart valve implant as viewed from the aortic sinus; and

FIGS. 4-5 illustrate selected aspects of the basilica procedure;

FIG. 6 schematically illustrates a second replacement heart valve implant being implanted within the first replacement heart valve implant after the basilica procedure;

FIG. 7 is a schematic view illustrating selected aspects related to implanting the second replacement heart valve implant within the first replacement heart valve implant after the basilica procedure as viewed from the aortic sinus;

FIGS. 8-9 illustrate selected aspects of an all-in-one basilica system according to the disclosure; and

FIGS. 10-13 illustrate selected aspects of the basilica procedure using the all-in-one basilica system of the disclosure.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently-such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

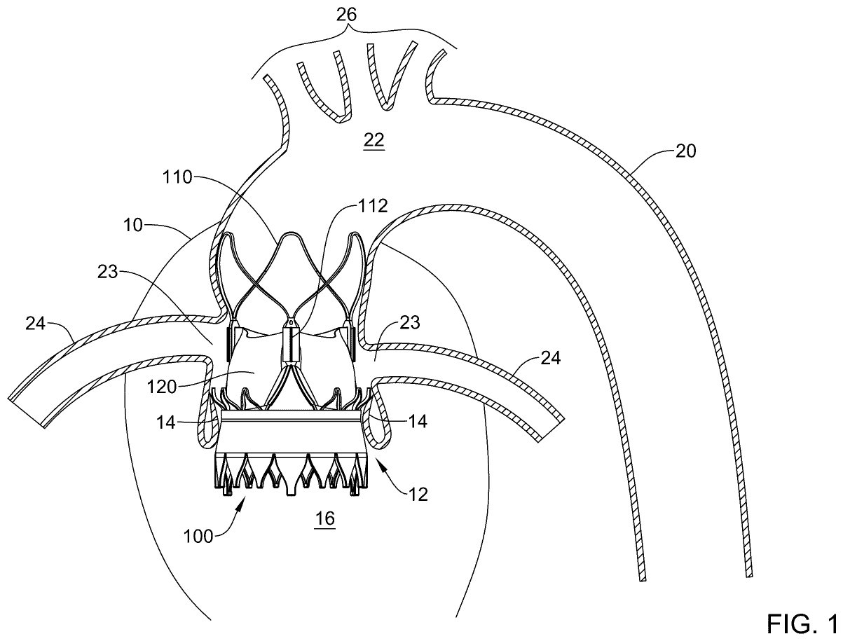

FIG. 1 illustrates a schematic partial cut-away view of a portion of a patient's heart 10 including the aortic valve 12 having native valve leaflets 14 disposed within and/or extending from the native valve annulus, the left ventricle 16, and certain connected vasculature, such as the aorta 20 connected to the aortic valve 12 of the patient's heart 10 by the aortic arch 22 and the ascending aorta, the coronary ostia 23 of the coronary arteries 24, which extend from the aortic sinuses and/or the ascending aorta, and other large arteries 26 (e.g., subclavian and/or carotid arteries, etc.) that extend from the aortic arch 22 to important internal organs. For the purpose of this disclosure, the discussion herein is directed toward treating the aortic valve 12 and will be so described in the interest of brevity. This, however, is not intended to be limiting as the skilled person will recognize that the following discussion may also apply to other heart valves, vessels, and/or treatment locations within a patient.

FIG. 1 further illustrates selected aspects of a replacement heart valve implant 100 positioned within the aortic valve 12 and/or the native valve annulus of the aortic valve 12. It should be appreciated that the replacement heart valve implant 100 can be any type of replacement heart valve (e.g., a mitral valve, an aortic valve, etc.). In use, the replacement heart valve implant 100 may be implanted (e.g., such as through transcatheter delivery) in the aortic valve 12 of the heart 10. The replacement heart valve implant 100 can be configured to allow one-way flow through the replacement heart valve implant 100 from an inflow end to an outflow end.

The replacement heart valve implant 100 may include an expandable framework 110 defining a central lumen. Some suitable but non-limiting examples of materials that may be used to form the expandable framework 110, including but not limited to metals and metal alloys, composites, ceramics, polymers, and the like, are described below. The replacement heart valve implant 100 and/or the expandable framework 110 may be configured to shift between a radially collapsed configuration and a radially expanded configuration. In some embodiments, the expandable framework 110 may be self-expanding. In some embodiments, the expandable framework 110 may be self-biased toward the radially expanded configuration. In some embodiments, the expandable framework 110 may be mechanically expandable. In some embodiments, the expandable framework 110 may be balloon expandable.

In some embodiments, the replacement heart valve implant 100 may include a plurality of valve leaflets 120 disposed within the central lumen. The plurality of valve leaflets 120 may be coupled, secured, and/or fixedly attached to the expandable framework 110 at a plurality of commissures 112. The plurality of valve leaflets 120 may be configured to shift between an open position and a closed position. The plurality of valve leaflets 120 may be configured to substantially restrict fluid flow through the replacement heart valve implant 100 in the closed position. The plurality of valve leaflets 120 may move apart from each other in the open position to permit fluid flow through the replacement heart valve implant 100.

In some embodiments, the plurality of valve leaflets 120 may be comprised of a polymer, such as a thermoplastic polymer. In some embodiments, the plurality of valve leaflets 120 may include at least 50 percent by weight of a polymer. In some embodiments, the plurality of valve leaflets 120 may be formed from bovine pericardial or other living tissue. Other configurations and/or materials are also contemplated.

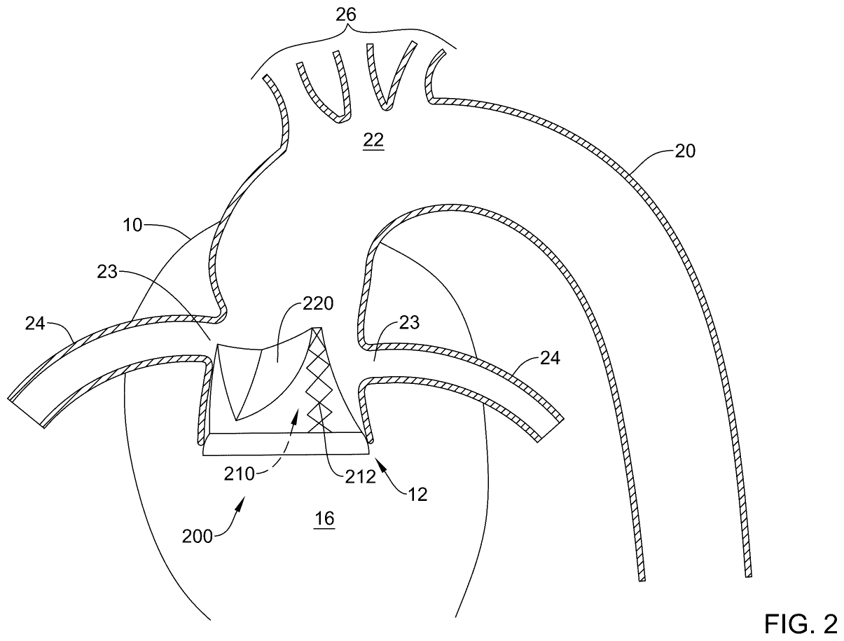

FIG. 2 illustrates selected aspects of a replacement heart valve implant 200 (e.g., a surgical replacement heart valve implant) positioned within the aortic valve 12 and/or the native valve annulus of the aortic valve 12. It should be appreciated that the replacement heart valve implant 200 can be any type of replacement heart valve (e.g., a mitral valve, an aortic valve, etc.). In use, the replacement heart valve implant 200 may be implanted (e.g., such as through surgical delivery) in the aortic valve 12 of the heart 10. In at least some surgical delivery procedures, the native valve leaflets may be excised, as shown in FIG. 2. The replacement heart valve implant 200 can be configured to allow one-way flow through the replacement heart valve implant 200 from an inflow end to an outflow end.

The replacement heart valve implant 200 may include an expandable framework 210 defining a central lumen. Some suitable but non-limiting examples of materials that may be used to form the expandable framework 210, including but not limited to metals and metal alloys, composites, ceramics, polymers, and the like, are described below. The replacement heart valve implant 200 and/or the expandable framework 210 may be configured to shift between a radially collapsed configuration and a radially expanded configuration. In some embodiments, the expandable framework 210 may be self-expanding. In some embodiments, the expandable framework 210 may be self-biased toward the radially expanded configuration. In some embodiments, the expandable framework 210 may be mechanically expandable. In some embodiments, the expandable framework 210 may be balloon expandable.

In some embodiments, the replacement heart valve implant 200 may include a plurality of valve leaflets 220 coupled, secured, and/or fixedly attached to the expandable framework 210 at a plurality of commissures 212. The plurality of valve leaflets 220 may be configured to shift between an open position and a closed position. The plurality of valve leaflets 220 may be configured to substantially restrict fluid flow through the replacement heart valve implant 200 in the closed position. The plurality of valve leaflets 220 may move apart from each other in the open position to permit fluid flow through the replacement heart valve implant 200.

In some embodiments, the plurality of valve leaflets 220 may be comprised of a polymer, such as a thermoplastic polymer. In some embodiments, the plurality of valve leaflets 220 may include at least 50 percent by weight of a polymer. In some embodiments, the plurality of valve leaflets 220 may be formed from bovine pericardial or other living tissue. Other configurations and/or materials are also contemplated.

For the purpose of discussion, this disclosure describes features and/or procedures related to placement of a second replacement heart valve implant (e.g., the replacement heart valve implant 100, similar devices, etc.) delivered via a transcatheter procedure (although this is not strictly required) and implanted within a first replacement heart valve implant (e.g., the replacement heart valve implant 200, similar devices, etc.). As used herein, the terms “first replacement heart valve implant” (which may be used interchangeably herein with “initial replacement heart valve implant” or “original replacement heart valve implant”) and “second replacement heart valve implant” are intended to denote order of implantation or placement. As illustrated, the figures show a transcatheter replacement heart valve implant (e.g., a “TAV” device) as the second replacement heart valve implant disposed and/or implanted within a surgical replacement heart valve implant (e.g., an “SAV” device) as the first replacement heart valve implant. This is not intended to be limiting and the devices may be reversed and/or interchanged in any combination (e.g., an SAV device within a TAV device, a TAV device within a TAV device, etc.) within the scope of this disclosure.

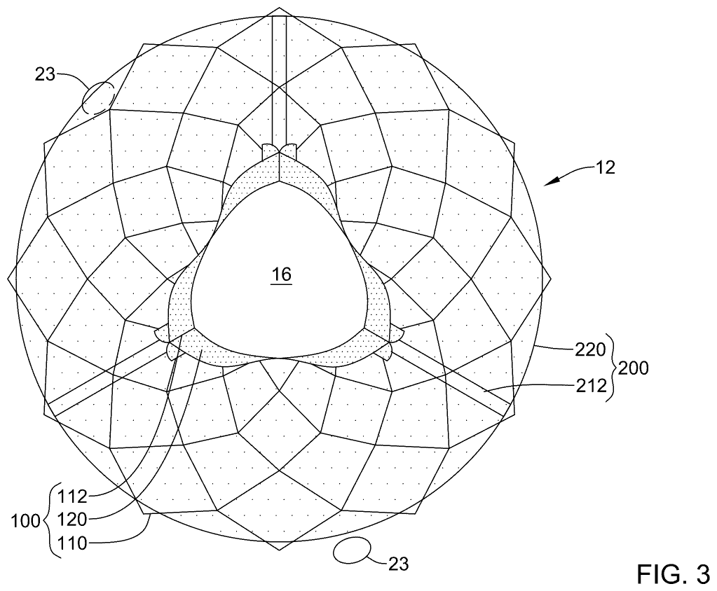

FIG. 3 is a top-down view of the area of the aortic valve 12 as seen from the ascending aorta and looking toward the left ventricle 16. In FIG. 3, the first replacement heart valve implant (e.g., the replacement heart valve implant 200) is disposed within the aortic valve 12 with the plurality of valve leaflets 220 of the first replacement heart valve implant (e.g., the replacement heart valve implant 200) urged radially outward by the second replacement heart valve implant (e.g., the replacement heart valve implant 100) which is disposed within the first replacement heart valve implant (e.g., the replacement heart valve implant 200). The plurality of commissures 212 of the first replacement heart valve implant (e.g., the replacement heart valve implant 200) are shown schematically as rectangular features. The expandable framework 110 of the second replacement heart valve implant (e.g., the replacement heart valve implant 100) may be seen over and/or within the plurality of valve leaflets 220 of the first replacement heart valve implant (e.g., the replacement heart valve implant 200). The plurality of valve leaflets 120 of the second replacement heart valve implant (e.g., the replacement heart valve implant 100) is shown inside the first replacement heart valve implant (e.g., the replacement heart valve implant 200) attached to the expandable framework 110 of the second replacement heart valve implant (e.g., the replacement heart valve implant 100) at the plurality of commissures 112. To improve clarity, the plurality of valve leaflets 220 is shown with dot shading having a first density, and the plurality of valve leaflets 120 is shown with dot shading having a second density greater than the first density. The plurality of valve leaflets 120 is shown in the open position such that the replacement heart valve is “open” and/or permits flow therethrough.

As may be seen at the upper left of FIG. 3, one of the coronary ostia 23 is at least partially obstructed from view. The anatomy of patients may vary from patient to patient. In some cases, one or both of the coronary ostia 23 may be partially or completely obstructed from view, which may make locating and/or accessing the coronary ostia 23 more difficult and/or even impossible. When placed properly, the commissures of a replacement heart valve implant are usually disposed at a position offset circumferentially from the coronary ostia 23 (e.g., the coronary ostia 23 are disposed between adjacent commissures), as shown in FIG. 3. In some cases, blood may still get to and/or flow into the coronary ostia 23 even if they are partially or completely obstructed by the plurality of valve leaflets 220 of the first replacement heart valve implant (e.g., the replacement heart valve implant 200) due to the shape and/or configuration of the aortic sinus.

Bench testing using several commercially available replacement heart valve implants has been performed to analyze the risk associated with replacement heart valve implant in replacement heart valve implant procedures. Two different commercially available replacement heart valve implants (e.g., first implant A and first implant B) were evaluated as the first replacement heart valve implant at several different sinotubular junction (STJ) heights (e.g., 15 mm and 20 mm) to consider the effect of anatomical differences using the same commercially available replacement heart valve implant (e.g., second implant C) as the second replacement heart valve implant. The second replacement heart valve implant was placed at two different relative heights (e.g., low and high) within each first replacement heart valve implant to represent variations in surgeon placement. It is noted that each implant used in the testing is different, and no combination placed the same type of valve inside of itself (e.g., first implant A was not placed inside first implant A).

Bench testing indicated that accessing the coronary ostia 23 is feasible in about 25% of valve combinations where no leaflet modification is made. The combinations and feasibility were tested as follows (Table 1):

| STJ | First | Second | Left Coronary | Right Coronary |
|---|---|---|---|---|
| height | valve | valve | Artery Access? | Artery Access? |
| 15 mm | A | C - low | Yes | Yes |
| 15 mm | A | C - high | No | No |
| 20 mm | A | C - low | Yes | Yes |
| 20 mm | A | C - high | No | No |
| 15 mm | B | C - low | No | No |
| 15 mm | B | C - high | No | No |
| 20 mm | B | C - low | No | No |
| 20 mm | B | C - high | No | No |

The bench testing was performed again using the same conditions after performing the basilica procedure to modify the leaflets of the first replacement heart valve implant prior to implanting the second replacement heart valve implant. The combinations and feasibility of accessing the coronary ostia 23 were evaluated as follows (Table 2):

| STJ | First | Second | Left Coronary | Right Coronary |
|---|---|---|---|---|
| height | valve | valve | Artery Access? | Artery Access? |
| 15 mm | A | C - low | Yes | Yes |
| 15 mm | A | C - high | Yes | Yes |
| 20 mm | A | C - low | Yes | Yes |
| 20 mm | A | C - high | Yes | Yes |
| 15 mm | B | C - low | Yes | Yes |
| 15 mm | B | C - high | No | No |
| 20 mm | B | C - low | Yes | Yes |
| 20 mm | B | C - high | No | Yes |

As may be seen in Table 2, after performing the basilica procedure, the feasibility of accessing the coronary ostia 23 after implanting the second replacement heart valve implant within the first replacement heart valve implant rose to 82%. Of particular note, the feasibility of access using first implant A rose from 50% to 100%, and the feasibility of access using first implant B rose from 0% to 62%. In view of the testing, the basilica procedure may be considered to provide substantially improved access to the coronary ostia 23.

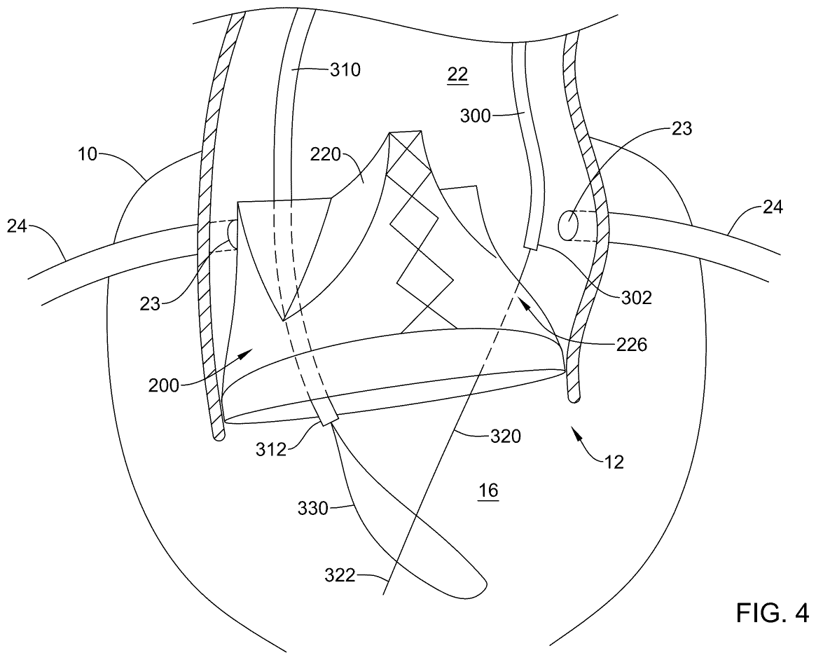

FIGS. 4-6 schematically illustrate the basilica procedure. FIG. 4 shows the first replacement heart valve implant (e.g., the replacement heart valve implant 200) disposed within the aortic valve 12. The basilica procedure involves advancing a first catheter 300 through the patient's vasculature until a distal end 302 of the first catheter 300 is disposed adjacent the first replacement heart valve implant (e.g., the replacement heart valve implant 200) outside of the plurality of valve leaflets 220. A second catheter 310 is advanced through the patient's vasculature until a distal end 312 of the second catheter 310 is advanced into and/or through the first replacement heart valve implant (e.g., the replacement heart valve implant 200) inside of the plurality of valve leaflets 220. A conductive guidewire 320 is advanced out of the first catheter 300 and pushed through a first leaflet of the plurality of valve leaflets 220 at a puncture site 226 such that a distal end 322 of the conductive guidewire 320 is disposed inside the plurality of valve leaflets 220 and/or upstream of the first replacement heart valve implant (e.g., the replacement heart valve implant 200). A snare 330 is advanced out of the second catheter 310 to a position adjacent the distal end 322 of the conductive guidewire 320, as seen in FIG. 4.

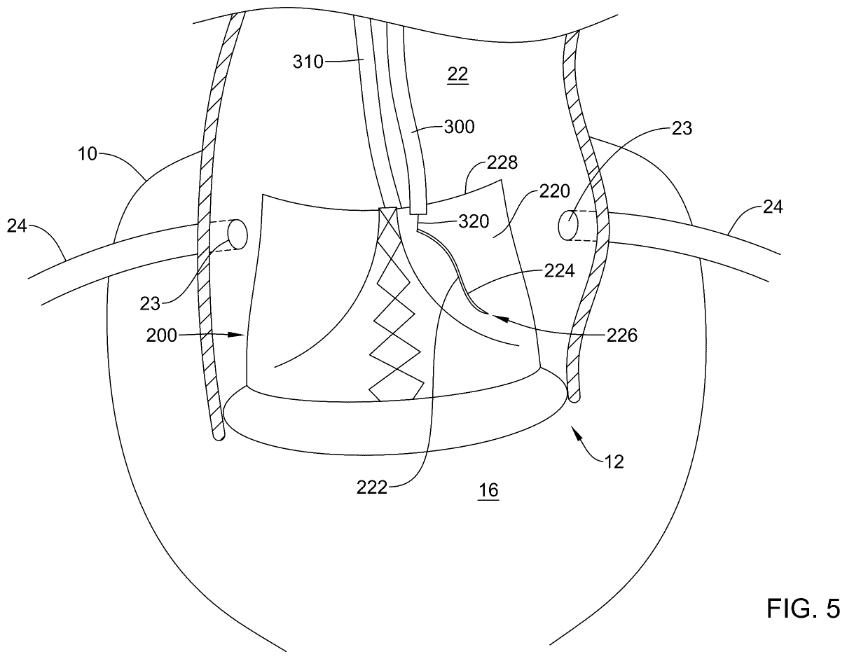

Next, the snare 330 is tightened around the distal end 322 of the conductive guidewire 320 and RF energy is applied to the snare 330 and/or the conductive guidewire 320. The snare 330 is maintained inside the plurality of valve leaflets 220 and the conductive guidewire 320 extends through the first leaflet of the plurality of valve leaflets 220. The conductive guidewire 320 (and/or the first catheter 300) and/or the snare 330 (and/or the second catheter 310) are pulled proximally while RF energy is applied to the snare 330 and/or the conductive guidewire 320, thereby lacerating the first leaflet of the plurality of valve leaflets 220 from the puncture site 226 to a free edge 228 of the first leaflet of the plurality of valve leaflets 220 to form a first edge 222 and a second edge 224 opposing the first edge 222 along the laceration, as shown in FIG. 5. If desired, the process may be repeated in a second leaflet of the plurality of valve leaflets 220.

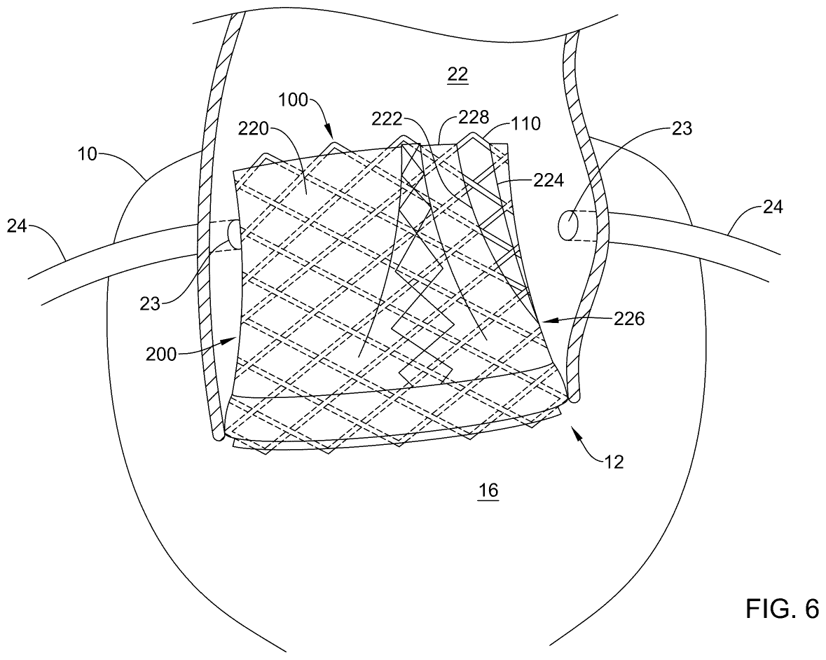

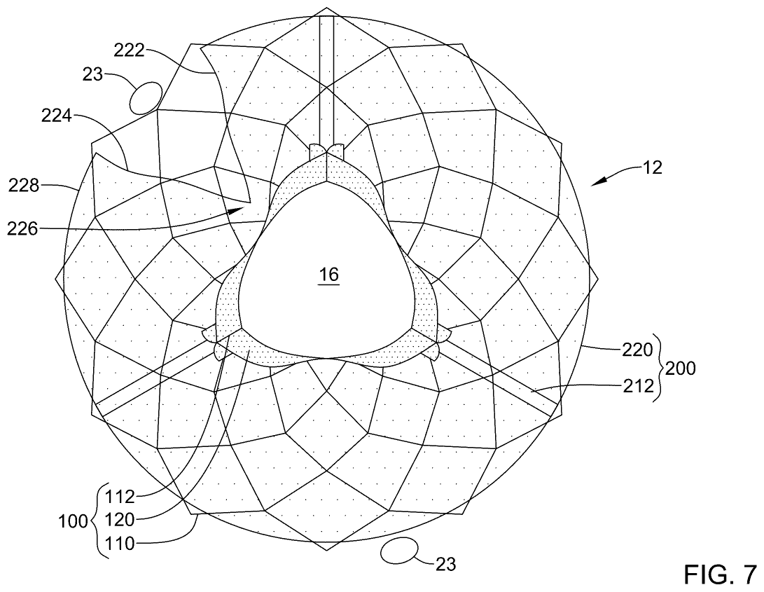

FIG. 6 illustrates the second replacement heart valve implant (e.g., the replacement heart valve implant 100) being deployed within the first replacement heart valve implant (e.g., the replacement heart valve implant 200). In FIG. 6, the expandable framework 110 of the second replacement heart valve implant (e.g., the replacement heart valve implant 100) is not fully expanded. As seen in FIG. 6, the first edge 222 and the second edge 224 have begun to move apart from each other to form an opening through the first leaflet of the plurality of valve leaflets 220 from the puncture site 226 to the free edge 228 of the first leaflet of the plurality of valve leaflets 220 as the second replacement heart valve implant (e.g., the replacement heart valve implant 100) is expanded. If the laceration is formed and/or placed correctly, the opening between the first edge 222 and the second edge 224 will overlap and/or coincide with the ostium 23 of the coronary artery 24 disposed adjacent thereto, thereby providing access to the ostium 23 of the coronary artery 24 disposed adjacent thereto, as shown in FIG. 7, when the expandable framework 110 of the second replacement heart valve implant (e.g., the replacement heart valve implant 100) is expanded within the first replacement heart valve implant (e.g., the replacement heart valve implant 200).

However, the basilica procedure is not without difficulty. For example, the use of “standard” catheters may make precisely locating the distal end 302 of the first catheter 300 and/or the conductive guidewire 320 relative to the first leaflet of the plurality of valve leaflets 220 of the first replacement heart valve implant (e.g., the replacement heart valve implant 200) challenging. In some instances, the conductive guidewire 320 may be pushed through the first leaflet of the plurality of valve leaflets 220 (e.g., the puncture site 226 may be located) at an off-center location (e.g., closer to one of the plurality of commissures 212, rather than centered between them), which may lead to and/or cause the laceration to be shifted circumferentially from its desired location and/or from the ostium 23 of the coronary artery 24 disposed adjacent thereto. This could cause the ostium 23 of the coronary artery 24 disposed adjacent to the first leaflet of the plurality of valve leaflets 220 to remain partially or completely obstructed, even after the basilica procedure is performed. Accordingly, it is desirable to develop and/or use an easy-to-use, all-in-one basilica system in accordance with the disclosure so as to improve the reliability and/or accuracy of positioning the system and/or components thereof, and/or to improve the efficacy of the procedure, while requiring less training, less skill, and/or less time to perform the procedure.

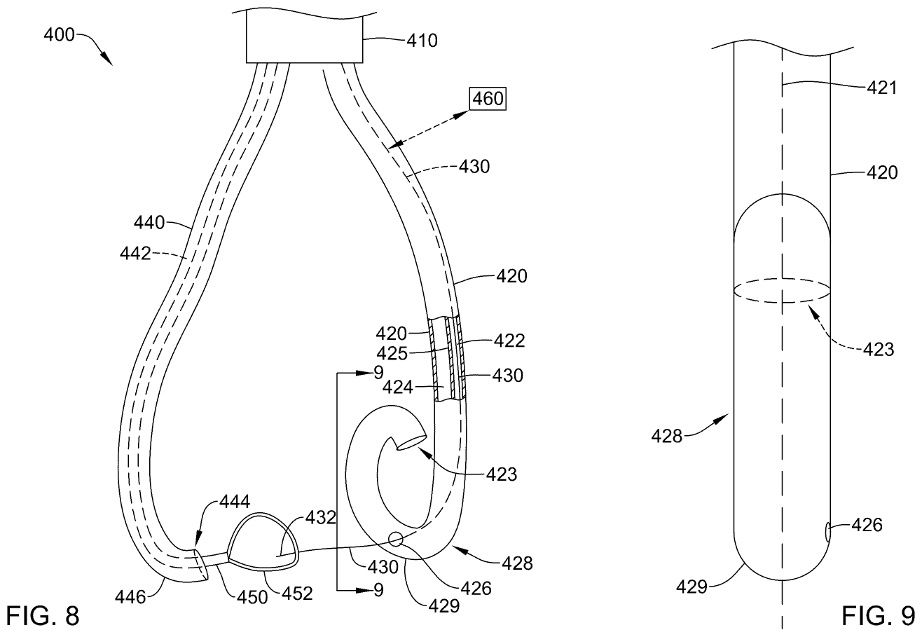

FIG. 8 illustrates selected aspects of a basilica system 400 for use in replacement heart valve implant procedures in accordance with the disclosure. In some embodiments, the basilica system 400 may comprise a delivery catheter 410. In at least some embodiments, the delivery catheter 410 may include a lumen extending proximally from a distal end of the delivery catheter 410. In some embodiments, the delivery catheter 410 may include a plurality of lumens extending proximally from the distal end of the delivery catheter 410. In some embodiments, the lumen or the plurality of lumens may extend proximally from the distal end of the delivery catheter 410 to a proximal end of the delivery catheter 410. Other configurations are also contemplated.

In some embodiments, the basilica system 400 may comprise a first catheter 420. In some embodiments, the first catheter 420 may include and/or may be a pigtail catheter. In some embodiments, the first catheter 420 and/or the pigtail catheter may include a first lumen 422 extending longitudinally therein. In some embodiments, the first catheter 420 and/or the pigtail catheter may be slidably disposed within the delivery catheter 410.

In some embodiments, the first catheter 420 and/or the pigtail catheter may include a second lumen 424 extending longitudinally therein. In some embodiments, the first catheter 420 and/or the pigtail catheter may include an internal wall 425 at least partially defining the first lumen 422 and/or the second lumen 424. In some embodiments, the internal wall 425 may separate the first lumen 422 from the second lumen 424. In some alternative configurations, the first lumen 422 may be a separate and/or independent tubular member disposed within the second lumen 424 and/or the delivery catheter 410.

The first catheter 420 and/or the pigtail catheter may include a distal tip 423 disposed at a distal end of the first catheter 420 and/or the pigtail catheter. In some embodiments, the second lumen 424 may extend proximally from the distal tip 423. The first catheter 420 and/or the pigtail catheter may include a side port 426 disposed proximal of the distal tip 423. In at least some embodiments, the first lumen 422 may extend proximally from the side port 426.

In some embodiments, the first catheter 420 and/or the pigtail catheter may include a preformed recurve portion 428 disposed proximal of the distal tip 423 of the first catheter 420 and/or the pigtail catheter in an unconstrained configuration and/or in a deployed configuration. In some embodiments, when the first catheter 420 and/or the pigtail catheter is unconstrained, the first catheter 420 and/or the pigtail catheter may assume the deployed configuration. The first catheter 420 and/or the pigtail catheter may include an elongate portion extending proximally from the preformed recurve portion 428. In some embodiments, the side port 426 may be disposed in the preformed recurve portion 428 of the first catheter 420 and/or the pigtail catheter when the first catheter 420 and/or the pigtail catheter is in the unconstrained configuration and/or the deployed configuration. In some embodiments, the side port 426 may be disposed proximate a base 429 of the preformed recurve portion 428. In the unconstrained configuration and/or the deployed configuration, the base 429 of the preformed recurve portion 428 may form and/or may be the distalmost extent of the first catheter 420 and/or the pigtail catheter. As such, in the unconstrained configuration and/or the deployed configuration, the base 429 of the preformed recurve portion 428 may be disposed distal of the distal tip 423 of the first catheter 420 and/or the pigtail catheter.

In some embodiments, the preformed recurve portion 428 may be shaped and/or configured to align with a cusp of a leaflet of the replacement heart valve implant (e.g., the first replacement heart valve implant and/or the replacement heart valve implant 200). In some embodiments, the base 429 of the preformed recurve portion 428 may be shaped and/or configured to align with and/or to engage with a cusp of a leaflet of the replacement heart valve implant (e.g., the first replacement heart valve implant and/or the replacement heart valve implant 200).

In some embodiments, the preformed recurve portion 428 may assume a generally planar configuration in the unconstrained configuration and/or the deployed configuration. For example, a centerline of the preformed recurve portion 428 may be generally disposed within a plane 421, as shown in FIG. 9. The side port 426 may open generally perpendicular to the plane 421. In some alternative configurations, the side port 426 may open at an oblique angle to the plane 421.

Returning now to FIG. 8, the basilica system 400 may comprise a conductive guidewire 430 slidably disposed within the first lumen 422 of the first catheter 420 and/or the pigtail catheter. The conductive guidewire 430 may be configured to be advanced out the side port 426 of the first catheter 420 and/or the pigtail catheter proximate a leaflet of the replacement heart valve implant (e.g., the first replacement heart valve implant and/or the replacement heart valve implant 200) disposed within the native heart valve of the patient. In some embodiments, the conductive guidewire 430 may extend to a proximal end of the delivery catheter 410. In some embodiments, the conductive guidewire 430 may extend proximally within the first lumen 422 of the first catheter 420 and/or the pigtail catheter to a proximal guidewire port or to an electrical connection point. In at least some embodiments, the conductive guidewire 430 may be configured to be electrically coupled to a source of RF energy 460. In at least some embodiments, the conductive guidewire 430 may include a distal end 432 configured to puncture the leaflet of the replacement heart valve implant (e.g., the first replacement heart valve implant and/or the replacement heart valve implant 200). As such, the conductive guidewire 430 may be devoid of an atraumatic distal tip or other similar features.

The basilica system 400 may comprise a second catheter 440. In some embodiments, the second catheter 440 may include and/or may be a snare catheter. In some embodiments, the second catheter 440 and/or the snare catheter may include a lumen 442 extending longitudinally therein to a distal end 444 of the second catheter 440 and/or the snare catheter. In some embodiments, the second catheter 440 and/or the snare catheter may be slidably disposed within the delivery catheter 410. In some embodiments, the second catheter 440 and/or the snare catheter may be slidably disposed within the delivery catheter 410 outside of and/or external to the first catheter 420 and/or the pigtail catheter (e.g., the second catheter 440 and/or the snare catheter is not disposed within and/or inside of the first catheter 420 and/or the pigtail catheter). In at least some embodiments, the second catheter 440 and/or the snare catheter may be slidable relative to the delivery catheter 410 independently of the first catheter 420 and/or the pigtail catheter, and vice versa.

In at least some embodiments, the second catheter 440 and/or the snare catheter may include a distal portion having a pre-shaped bend 446 in an unconstrained configuration and/or in a deployed configuration. In some embodiments, when the second catheter 440 and/or the snare catheter is unconstrained, the second catheter 440 and/or the snare catheter may assume the deployed configuration. The second catheter 440 and/or the snare catheter may include an elongate portion extending proximally from the pre-shaped bend 446. In some embodiments, the pre-shaped bend 446 may be disposed proximal of the distal end 444 of the second catheter 440 and/or the snare catheter.

In some embodiments, the pre-shaped bend 446 may be configured to orient the distal end 444 of the second catheter 440 and/or the snare catheter toward the side port 426 of the first catheter 420 and/or the pigtail catheter. For example, the distal end 444 of the second catheter 440 and/or the snare catheter may open toward the side port 426 of the first catheter 420 and/or the pigtail catheter in the unconstrained configuration and/or in the deployed configuration. In some embodiments, the pre-shaped bend 446 may orient the distal end 444 of the second catheter 440 and/or the snare catheter at an oblique angle relative to a longitudinal axis of the second catheter 440 and/or the snare catheter in the unconstrained configuration and/or in the deployed configuration.

The basilica system 400 may comprise snare shaft 450 slidably disposed within the lumen 442 of the second catheter 440 and/or the snare catheter. In some embodiments, the snare shaft 450 may extend proximally within the second catheter 440 and/or the snare catheter to a proximal end of the second catheter 440 and/or the snare catheter. In some embodiments, the snare shaft 450 may extend to a proximal port or to an electrical connection point. In some embodiments, the snare shaft 450 may be configured to be electrically coupled to the source of RF energy 460.

In some embodiments, the snare shaft 450 may comprise and/or may be formed from a nonconductive material. In some embodiments, the snare shaft 450 may comprise a nonconductive coating disposed on an outer surface thereof. Other configurations are also contemplated. In some embodiments, the snare shaft 450 may comprise a wire loop 452 extending from a distal end of the snare shaft 450. In at least some embodiments, the wire loop 452 may be formed from an electrically conductive material. In some embodiments, the wire loop 452 may extend proximally within the snare shaft 450 to a proximal end of the snare shaft 450 and/or to the electrical connection point. In some embodiments, the wire loop 452 may be configured to be electrically coupled to the source of RF energy 460.

The wire loop 452 may be configured to grasp the distal end 432 of the conductive guidewire 430. In some embodiments, grasping the distal end 432 of the conductive guidewire 430 with the wire loop 452 may close and/or complete an electrical pathway electrically coupled to the source of RF energy 460. As discussed herein, during use, the source of RF energy may be activated to apply RF energy to the conductive guidewire 430. The wire loop 452 may be axially movable relative to the snare shaft 450 to facilitate closing the wire loop 452 around the distal end 432 of the conductive guidewire 430 in order to grasp the distal end 432 of the conductive guidewire 430. Once the distal end 432 of the conductive guidewire 430 has been grasped by the wire loop 452, manipulation of the snare shaft 450 may move, translate, shift, etc. the conductive guidewire 430 relative to the second catheter 440 and/or the snare catheter. In some embodiments, once the distal end 432 of the conductive guidewire 430 has been grasped by the wire loop 452, the snare shaft 450 may be configured to pull the distal end 432 of the conductive guidewire 430 into the distal end 444 and/or into the lumen 442 of the second catheter 440 and/or the snare catheter.

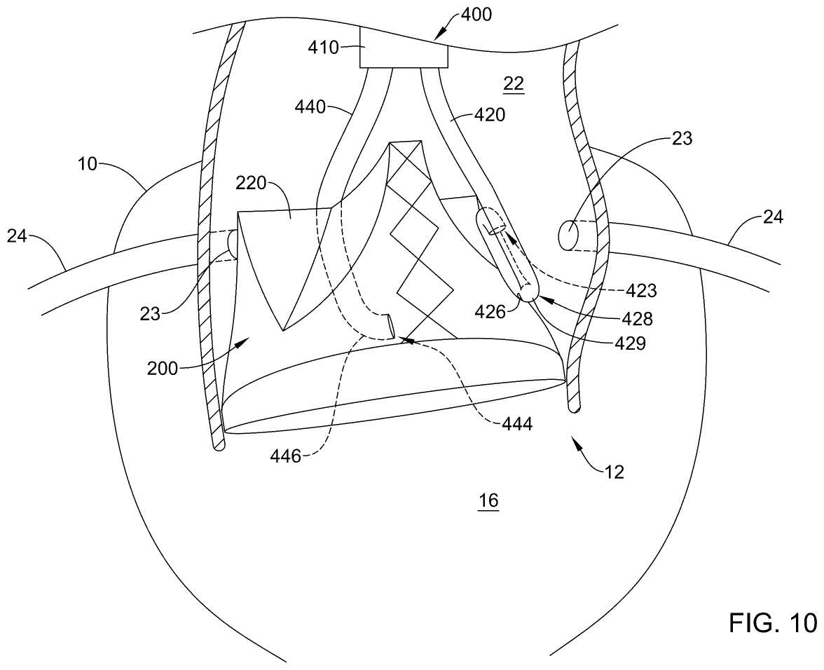

FIGS. 10-13 illustrate selected aspects related to the use of the basilica system in accordance with the disclosure. A method of splitting a leaflet (e.g., a first leaflet of the plurality of valve leaflets 220) of a replacement heart valve implant (e.g., the first replacement heart valve implant and/or the replacement heart valve implant 200) disposed within a native heart valve (e.g., the aortic valve 12) of a patient may comprise advancing the delivery catheter 410 to a position adjacent the replacement heart valve implant (e.g., the first replacement heart valve implant and/or the replacement heart valve implant 200). In at least some embodiments, the method may comprise advancing the delivery catheter 410 percutaneously and/or intravascularly to a position adjacent the replacement heart valve implant (e.g., the first replacement heart valve implant and/or the replacement heart valve implant 200), as seen in FIG. 10.

The method of splitting the leaflet may comprise advancing the first catheter 420 and/or the pigtail catheter distally out of the delivery catheter 410 to a position adjacent an outer surface of the leaflet (e.g., the first leaflet of the plurality of valve leaflets 220) of the replacement heart valve implant (e.g., the first replacement heart valve implant and/or the replacement heart valve implant 200), as seen in FIG. 10. In at least some embodiments, when and/or as the first catheter 420 and/or the pigtail catheter is advanced distally out of the delivery catheter 410 to the position adjacent the outer surface of the leaflet (e.g., the first leaflet of the plurality of valve leaflets 220) of the replacement heart valve implant (e.g., the first replacement heart valve implant and/or the replacement heart valve implant 200), the first catheter 420 and/or the pigtail catheter may assume and/or may shift into the deployed configuration and/or the unconstrained configuration.

As discussed herein, the first catheter 420 and/or the pigtail catheter may comprise a preformed recurve portion 428 disposed proximal the distal tip 423 in the deployed configuration and/or the unconstrained configuration. In at least some embodiments, advancing the first catheter 420 and/or the pigtail catheter distally out of the delivery catheter 410 to the position adjacent the outer surface of the leaflet (e.g., the first leaflet of the plurality of valve leaflets 220) of the replacement heart valve implant (e.g., the first replacement heart valve implant and/or the replacement heart valve implant 200) further comprises positioning the preformed recurve portion 428 of the first catheter 420 and/or the pigtail catheter in a cusp of the leaflet (e.g., the first leaflet of the plurality of valve leaflets 220) of the replacement heart valve implant (e.g., the first replacement heart valve implant and/or the replacement heart valve implant 200) with the side port 426 facing towards the outer surface of the leaflet (e.g., the first leaflet of the plurality of valve leaflets 220) of the replacement heart valve implant (e.g., the first replacement heart valve implant and/or the replacement heart valve implant 200).

The method of splitting the leaflet may comprise advancing the second catheter 440 and/or the snare catheter distally out of the delivery catheter 410 to a position adjacent an inner surface of the leaflet (e.g., the first leaflet of the plurality of valve leaflets 220) of the replacement heart valve implant (e.g., the first replacement heart valve implant and/or the replacement heart valve implant 200), as seen in FIG. 10. In some embodiments, the method may comprise advancing the distal end 444 of the second catheter 440 and/or the snare catheter distally out of the delivery catheter 410 to the position adjacent the inner surface of the leaflet (e.g., the first leaflet of the plurality of valve leaflets 220) of the replacement heart valve implant (e.g., the first replacement heart valve implant and/or the replacement heart valve implant 200). In some embodiments, the method may comprise advancing the second catheter 440 and/or the snare catheter distally out of the delivery catheter 410 into a central lumen and/or an interior of the replacement heart valve implant (e.g., the first replacement heart valve implant and/or the replacement heart valve implant 200). In at least some embodiments, when and/or as the second catheter 440 and/or the snare catheter is advanced distally out of the delivery catheter 410 to the position adjacent the inner surface of the leaflet (e.g., the first leaflet of the plurality of valve leaflets 220) of the replacement heart valve implant (e.g., the first replacement heart valve implant and/or the replacement heart valve implant 200), the second catheter 440 and/or the snare catheter may assume and/or may shift into the deployed configuration and/or the unconstrained configuration.

As discussed herein, the distal portion of the second catheter 440 and/or the snare catheter may comprise a pre-shaped bend 446 configured to orient the distal end 444 of the second catheter 440 and/or the snare catheter toward the side port 426 of the first catheter 420 and/or the pigtail catheter.

In some embodiments, in the deployed configuration and/or the unconstrained configuration, a distalmost extent of the pre-shaped bend 446 of the second catheter 440 and/or the snare catheter, as measured axially from a distal end of the delivery catheter 410, may be within about 10% of a distalmost extent of the base 429 of the preformed recurve portion 428 of the first catheter 420 and/or the pigtail catheter, as measured axially from the distal end of the delivery catheter 410. In some embodiments, in the deployed configuration and/or the unconstrained configuration, the distalmost extent of the pre-shaped bend 446 of the second catheter 440 and/or the snare catheter, as measured axially from the distal end of the delivery catheter 410, may be within about 7.5% of the distalmost extent of the base 429 of the preformed recurve portion 428 of the first catheter 420 and/or the pigtail catheter, as measured axially from the distal end of the delivery catheter 410. In some embodiments, in the deployed configuration and/or the unconstrained configuration, the distalmost extent of the pre-shaped bend 446 of the second catheter 440 and/or the snare catheter, as measured axially from the distal end of the delivery catheter 410, may be within about 5% of the distalmost extent of the base 429 of the preformed recurve portion 428 of the first catheter 420 and/or the pigtail catheter, as measured axially from the distal end of the delivery catheter 410. In some embodiments, in the deployed configuration and/or the unconstrained configuration, the distalmost extent of the pre-shaped bend 446 of the second catheter 440 and/or the snare catheter, as measured axially from the distal end of the delivery catheter 410, may be within about 2.5% of the distalmost extent of the base 429 of the preformed recurve portion 428 of the first catheter 420 and/or the pigtail catheter, as measured axially from the distal end of the delivery catheter 410. Other configurations are also contemplated.

In some embodiments, the first catheter 420 and/or the pigtail catheter and the second catheter 440 and/or the snare catheter may include markings configured to identify a relative position of the first catheter 420 and/or the pigtail catheter and the second catheter 440 and/or the snare catheter with respect to the delivery catheter 410.

In some embodiments, the first catheter 420 and/or the pigtail catheter and the second catheter 440 and/or the snare catheter may include one or more features disposed proximate a proximal end of the delivery catheter 410 configured to secure the first catheter 420 and/or the pigtail catheter relative to the second catheter 440 and/or the snare catheter. For example, the first catheter 420 and/or the pigtail catheter and the second catheter 440 and/or the snare catheter may include one or more latches, securement members, etc. configured to prevent relative axial movement between the first catheter 420 and/or the pigtail catheter and the second catheter 440 and/or the snare catheter once engaged.

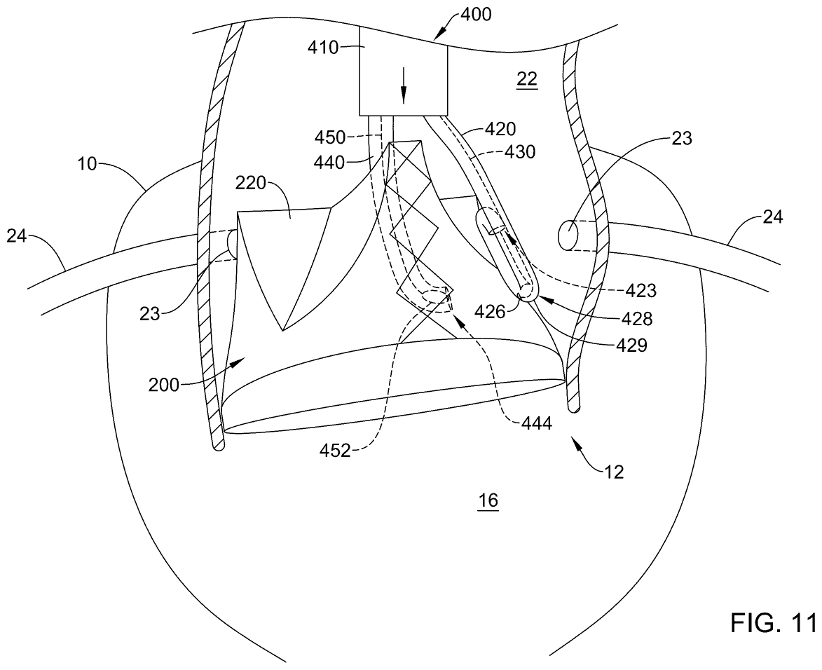

The method of splitting the leaflet may comprise, after advancing the first catheter 420 and/or the pigtail catheter to the position adjacent the outer surface of the leaflet and after advancing the second catheter 440 and/or the snare catheter to the position adjacent the inner surface of the leaflet, advancing the delivery catheter 410 distally relative to the first catheter 420 and/or the pigtail catheter and the second catheter 440 and/or the snare catheter to urge the distal end 444 of the second catheter 440 and/or the snare catheter toward the side port 426 formed in the preformed recurve portion 428 of the first catheter 420 and/or the pigtail catheter proximal of the distal tip 423 of the first catheter 420 and/or the pigtail catheter, as seen in FIG. 11. In some embodiments, when the first catheter 420 and/or the pigtail catheter is in the deployed configuration and the second catheter 440 and/or the snare catheter is in the deployed configuration, distal advancement of the delivery catheter 410 relative to the first catheter 420 and/or the pigtail catheter and the second catheter 440 and/or the snare catheter may urge, move, translate, etc. the distal end 444 of the second catheter 440 and/or the snare catheter closer to the side port 426 formed in the preformed recurve portion 428 of the first catheter 420 and/or the pigtail catheter than to the distal tip 423 of the first catheter 420 and/or the pigtail catheter.

The method of splitting the leaflet may comprise advancing the conductive guidewire 430 distally within and/or relative to the first catheter 420 and/or the pigtail catheter. The method may comprise advancing the conductive guidewire 430 distally out of the first catheter 420 and/or the pigtail catheter. In at least some embodiments, advancing the conductive guidewire 430 out of the first catheter 420 and/or the pigtail catheter may include advancing the conductive guidewire 430 out of and/or through the side port 426 of the first catheter 420 and/or the pigtail catheter. The method of splitting the leaflet may comprise advancing the conductive guidewire 430 through the leaflet (e.g., the first leaflet of the plurality of valve leaflets 220) of the replacement heart valve implant (e.g., the first replacement heart valve implant and/or the replacement heart valve implant 200) at a puncture site 226. In some embodiments, the method may comprise advancing the conductive guidewire 430 distally out of the first catheter 420 and/or the pigtail catheter and through the leaflet (e.g., the first leaflet of the plurality of valve leaflets 220) of the replacement heart valve implant (e.g., the first replacement heart valve implant and/or the replacement heart valve implant 200) at the puncture site 226 in a single movement or a single motion. In some alternative configurations, the method may comprise advancing the conductive guidewire 430 distally out of the first catheter 420 and/or the pigtail catheter and through the leaflet (e.g., the first leaflet of the plurality of valve leaflets 220) of the replacement heart valve implant (e.g., the first replacement heart valve implant and/or the replacement heart valve implant 200) at the puncture site 226 in a plurality of motions and/or a plurality of steps.

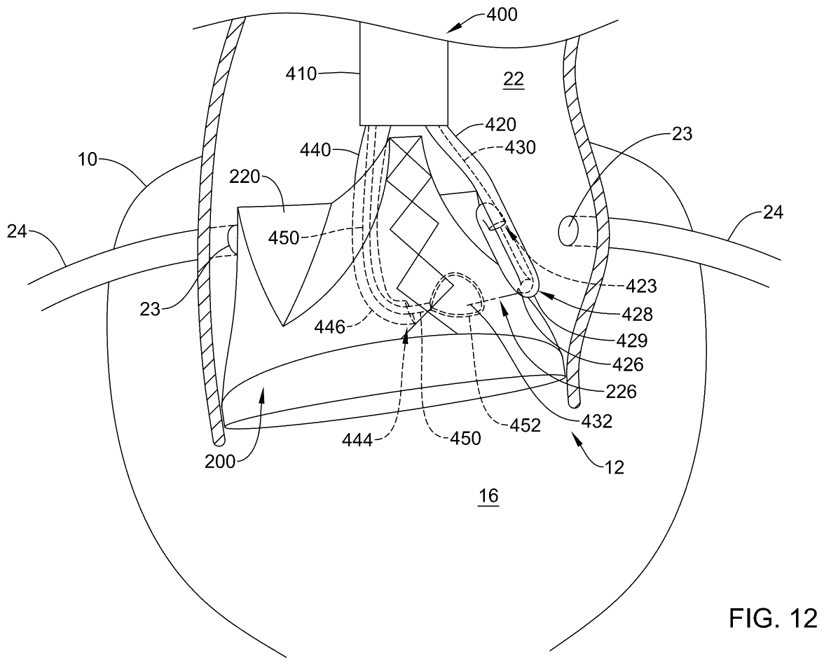

The method of splitting the leaflet may comprise grasping the conductive guidewire 430 with the wire loop 452 extending from the second catheter 440 and/or the snare catheter. In some embodiments, prior to grasping the conductive guidewire 430 with the wire loop 452, the method of splitting the leaflet may comprise advancing the snare shaft 450 and/or the wire loop 452 distally out of the second catheter 440 and/or the snare catheter within the central lumen and/or the interior of the replacement heart valve implant (e.g., the first replacement heart valve implant and/or the replacement heart valve implant 200). In some embodiments, the wire loop 452 may have a diameter of up to 20 millimeters in an open configuration. In some embodiments, advancing the conductive guidewire 430 distally out of the first catheter 420 and/or the pigtail catheter and through the leaflet (e.g., the first leaflet of the plurality of valve leaflets 220) includes advancing the distal end 432 of the conductive guidewire 430 into and/or through the wire loop 452, as seen in FIG. 12.

In some embodiments, when the distal end 444 of the second catheter 440 and/or the snare catheter is in close proximity to the side port 426 formed in the preformed recurve portion 428 of the first catheter 420 and/or the pigtail catheter, the side port 426 is configured to direct the distal end 432 of the conductive guidewire 430 toward the wire loop 452 when the conductive guidewire 430 is advanced distally relative to and/or out of the first catheter 420 and/or the pigtail catheter (e.g., via the side port 426).

In some embodiments, “close proximity” may be defined as about 0 millimeters to about 30 millimeters. In some embodiments, “close proximity” may be defined as about 0 millimeters to about 25 millimeters. In some embodiments, “close proximity” may be defined as about 0 millimeters to about 20 millimeters. In some embodiments, “close proximity” may be defined as about 0 millimeters to about 15 millimeters. In some embodiments, “close proximity” may be defined as about 0 millimeters to about 10 millimeters. In some embodiments, “close proximity” may be defined as about 1 millimeter to about 10 millimeters. In some embodiments, “close proximity” may be defined as about 1 millimeter to about 8 millimeters. In some embodiments, “close proximity” may be defined as about 1 millimeter to about 6 millimeters. In some embodiments, “close proximity” may be defined as about 2 millimeters to about 6 millimeters. Other configurations are also contemplated.

In some embodiments, the method of splitting the leaflet may comprise securing the first catheter 420 and/or the pigtail catheter and the second catheter 440 and/or the snare catheter relative to each other. In some embodiments, the method of splitting the leaflet may comprise securing the first catheter 420 and/or the pigtail catheter and the second catheter 440 and/or the snare catheter relative to the delivery catheter 410. In some embodiments, the method of splitting the leaflet may comprise securing the conductive guidewire 430 relative to the first catheter 420 and/or the pigtail catheter. In some embodiments, the method of splitting the leaflet may comprise securing the snare shaft 450 and/or the wire loop 452 relative to the second catheter 440 and/or the snare catheter.

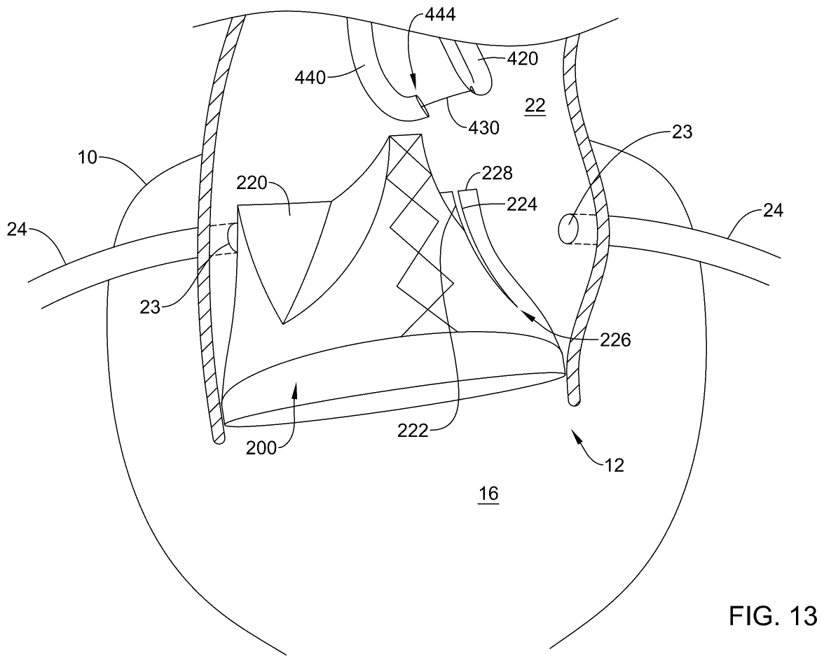

The method of splitting the leaflet may comprise pulling the conductive guidewire 430 proximally and/or downstream through the leaflet (e.g., the first leaflet of the plurality of valve leaflets 220) to lacerate the leaflet (e.g., the first leaflet of the plurality of valve leaflets 220) from the puncture site 226 to the free edge 228 of the leaflet (e.g., the first leaflet of the plurality of valve leaflets 220), as seen in FIG. 13.

In some embodiments, the method of splitting the leaflet may comprise applying RF energy to the conductive guidewire 430 while pulling the conductive guidewire 430 through the leaflet (e.g., the first leaflet of the plurality of valve leaflets 220) to lacerate the leaflet (e.g., the first leaflet of the plurality of valve leaflets 220). In some embodiments, the method of splitting the leaflet may comprise applying RF energy to the conductive guidewire 430 prior to pulling the conductive guidewire 430 through the leaflet (e.g., the first leaflet of the plurality of valve leaflets 220) to lacerate the leaflet (e.g., the first leaflet of the plurality of valve leaflets 220). In some embodiments, the method of splitting the leaflet may comprise applying RF energy to the conductive guidewire 430 prior to pulling the conductive guidewire 430 through the leaflet (e.g., the first leaflet of the plurality of valve leaflets 220) to lacerate the leaflet (e.g., the first leaflet of the plurality of valve leaflets 220) and then continuing to apply RF energy to the conductive guidewire 430 while pulling the conductive guidewire 430 through the leaflet (e.g., the first leaflet of the plurality of valve leaflets 220) to lacerate the leaflet (e.g., the first leaflet of the plurality of valve leaflets 220).

In some embodiments, the method of splitting the leaflet may comprise, prior to pulling the conductive guidewire 430 through the leaflet (e.g., the first leaflet of the plurality of valve leaflets 220) to lacerate the leaflet (e.g., the first leaflet of the plurality of valve leaflets 220), retracting the snare shaft 450 and/or the wire loop 452 into the second catheter 440 and/or the snare catheter to pull the distal end 432 of the conductive guidewire 430 into the lumen and/or the distal end 444 of the second catheter 440 and/or the snare catheter. In some embodiments, none of the wire loop 452 may be extended distal of the distal end 444 of the second catheter 440 and/or the snare catheter while pulling the conductive guidewire 430 through the leaflet (e.g., the first leaflet of the plurality of valve leaflets 220) to lacerate the leaflet (e.g., the first leaflet of the plurality of valve leaflets 220). In some situations, this may reduce the structure adjacent to and/or in contact with the leaflet (e.g., the first leaflet of the plurality of valve leaflets 220) and/or permit a smoother and/or cleaner laceration to be formed.

The materials that can be used for the various components of the system and the various elements thereof disclosed herein may include those commonly associated with medical devices. For simplicity purposes, the following discussion refers to the system. However, this is not intended to limit the devices, components, and methods described herein, as the discussion may be applied to other elements, members, components, or devices disclosed herein, such as, but not limited to, the delivery catheter, the first catheter and/or the pigtail catheter, the second catheter and/or the snare catheter, the snare shaft, the wire loop, the conductive guidewire, etc. and/or elements or components thereof.

In some embodiments, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively bright image on a fluoroscopy screen or another imaging technique (e.g., ultrasound, etc.) during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A basilica system for use in replacement heart valve implant procedures, comprising:
a pigtail catheter comprising a side port disposed proximal of a distal tip of the pigtail catheter;
a conductive guidewire slidably disposed within a first lumen of the pigtail catheter and configured to be advanced out the side port proximate a leaflet of a replacement heart valve implant disposed within a native heart valve of a patient;
a snare catheter comprising a lumen extending to a distal end of the snare catheter; and
a snare shaft slidably disposed within the lumen of the snare catheter, the snare shaft comprising a wire loop extending from a distal end of the snare shaft and configured to grasp a distal end of the conductive guidewire.

2. The basilica system of claim 1, wherein the pigtail catheter further comprises a second lumen extending proximally from the distal tip of the pigtail catheter;
wherein the first lumen extends proximally from the side port.

3. The basilica system of claim 1, wherein the pigtail catheter includes a preformed recurve portion proximal of the distal tip of the pigtail catheter in an unconstrained configuration.

4. The basilica system of claim 3, wherein the side port is disposed proximate a base of the preformed recurve portion.

5. The basilica system of claim 3, wherein a centerline of the preformed recurve portion is generally disposed within a plane and the side port opens generally perpendicular to the plane.

6. The basilica system of claim 1, wherein a distal portion of the snare catheter includes a pre-shaped bend in an unconstrained configuration, the pre-shaped bend being configured to orient the distal end of the snare catheter toward the side port of the pigtail catheter.

7. The basilica system of claim 6, wherein the pre-shaped bend orients the distal end of the snare catheter at an oblique angle relative to a longitudinal axis of the snare catheter.

8. The basilica system of claim 1, further comprising a delivery catheter, wherein the pigtail catheter and the snare catheter are slidably disposed within the delivery catheter.

9. The basilica system of claim 1, further comprising a source of RF energy electrically coupled to the conductive guidewire.

10. A basilica system for use in replacement heart valve implant procedures, comprising:
a delivery catheter;
a first catheter slidably disposed within the delivery catheter;
a second catheter slidably disposed within the delivery catheter;
a conductive guidewire slidably disposed within the first catheter and configured to be electrically coupled to a source of RF energy, the conductive guidewire including a distal end configured to puncture a leaflet of a replacement heart valve implant; and
a snare shaft slidably disposed within a lumen of the second catheter, the snare shaft comprising a wire loop extending from a distal end of the snare shaft and configured to grasp the distal end of the conductive guidewire.

11. The basilica system of claim 10, wherein in a deployed configuration, the first catheter comprises a preformed recurve portion disposed proximal the distal tip, and an elongate portion extending proximally from the preformed recurve portion;
wherein the first catheter comprises a side port disposed in the preformed recurve portion of the first catheter when the first catheter is in the deployed configuration.

12. The basilica system of claim 11, wherein the preformed recurve portion is shaped and configured to align with a cusp of the leaflet of the replacement heart valve implant.

13. The basilica system of claim 11, wherein in a deployed configuration, the second catheter includes a distal portion having a pre-shaped bend configured to orient a distal end of the second catheter toward the side port.

14. The basilica system of claim 13, wherein when the first catheter is in the deployed configuration and the second catheter is in the deployed configuration, distal advancement of the delivery catheter relative to the first catheter and the second catheter urges the distal end of the second catheter closer to the side port formed in the first catheter than to the distal tip of the first catheter.

15. The basilica system of claim 14, wherein when the distal end of the second catheter is in close proximity to the side port, the side port is configured to direct the conductive wire toward the wire loop when the conductive wire is advanced distally relative to the first catheter.

16. A method of splitting a leaflet of a replacement heart valve implant disposed within a native heart valve of a patient, comprising:
advancing a delivery catheter to a position adjacent the replacement heart valve implant;
advancing a first catheter distally out of the delivery catheter to a position adjacent an outer surface of the leaflet;
advancing a second catheter distally out of the delivery catheter to a position adjacent an inner surface of the leaflet;
advancing the delivery catheter distally relative to the first catheter and the second catheter to urge a distal end of the second catheter toward a side port formed in the first catheter proximal of a distal tip of the first catheter;
advancing a conductive guidewire out of the first catheter and through the leaflet at a puncture site;
grasping the conductive guidewire with a wire loop extending from the second catheter; and
pulling the conductive guidewire through the leaflet to lacerate the leaflet from the puncture site to a free edge of the leaflet.

17. The method of claim 16, further comprising applying RF energy to the conductive guidewire while pulling the conductive guidewire through the leaflet to lacerate the leaflet.

18. The method of claim 16, wherein advancing the conductive guidewire out of the first catheter and through the leaflet includes advancing a distal end of the conductive guidewire into the wire loop.

19. The method of claim 16, wherein the first catheter comprises a preformed recurve portion disposed proximal the distal tip in a deployed configuration, and wherein advancing the first catheter distally out of the delivery catheter to the position adjacent the outer surface of the leaflet further comprises positioning the preformed recurve portion in a cusp of the leaflet with the side port facing towards the leaflet.

20. The method of claim 16, further comprising:
prior to pulling the conductive guidewire through the leaflet to lacerate the leaflet, retracting the wire loop into the second catheter to pull a distal end of the conductive guidewire into the second catheter.
